(19) United States
(12) Patent Application Publication
(10) Pub. No.: US 2006/0188526 A1
(43) Pub. Date: Aug. 24, 2006

(54) Method for enhancing the immune response to Staphylococcus aureus infection

(76) Inventor: Kenji M. Cunnion, Norfolk, VA (US)

Correspondence Address: MILES & STOCKBRIDGE PC, 1751 PINNACLE DRIVE, SUITE 500, MCLEAN, VA, 22102-3833, US

(21) Appl. No.: 11/063,721

(22) Filed: Feb. 24, 2005

(51) Int. Cl. A61K 39/085; C12N 1/21

(52) U.S. Cl. 424/243.1; 435/252.3

(57) ABSTRACT

A method for enhancing the immune response of a patient to Staphylococcus aureus infection comprising administering to said patient an effective amount of agent that inhibits the cofactor that mediates (1) the removal of C3b by factor I from Staphylococcus aureus cells to which it has bonded by factor I and/or 2) the cleavage of C3b by factor I to forms inactive as opsonins for Staphylococcus aureus cells.

This work was supported by Public Health Service grant AI-01835 for the National Institute of Allergy and Infectious Diseases.

### BACKGROUND OF THE INVENTION

1. Field of the Invention

2. Description of the Prior Art

Opsonins are molecules which are capable, by virtue of being contemporaneously bound or attached to both a target and a phagocytic cell, of acting as a coupling agent between the target and the phagocytic cell to allow more efficient binding, engulfment, internalization and destruction of the target by the phagocytic cell. Naturally occurring opsonins can be categorized as belonging to the innate immune system (innate opsonins), which provides first line defense against foreign targets and microorganisms.

Families of opsonins include fragments of complement components C3, e.g., C3b and C4, collecting, and immunoglobulins. Other purported opsonins include fibronectin, alpha-2-macroglobulin (a2m), and C reactive protein (CRP).

Staphylococcus aureus is a frequent cause of severe hospital-acquired and community-acquired infections [Centers for Disease Control and Prevention. 2002. Public Health Dispatch: Vancomycin Resistant Staphylococcus aureus—Pennsylvania, 2002. Morb. Mortal. Wkly. Rep. 51: 902; Centers for Disease Control and Prevention. 2002. National Nosocomial Infections Surveillance (NNIS) system report, data summary from January 1992 to June 2002, issued August 2002. Am. J. Infect. Control. 30:458-475; Diekema, D. J., M. A. Pfaller, F. J. Schmitz, J. Smayevsky, J. Bell, R. N. Jones, M Beach. Survey of infections due to Staphylococcus species: frequency of occurrence and antimicrobial susceptibility of isolates collected in the United States, Canada, Latin America, Europe, and the Western Pacific region of the SENTRY Antimicrobial Surveillance Program, 1997-1999.2001. Clin. Infect. Dis. 32:S114 132].

Staphylococcal infections incur significant medical expense, and disability and death are common outcomes despite antibiotic treatment [Mylotte, J. M., C. McDermott, and J. A. Spooner. 1987. Prospective study of 114 consecutive episodes of Staphylococcus aureus bacteremia. Rev. Infect. Dis. 9:891 907; Rubin, R. J., C. A. Harrington, A. Poon, K. Dietrich, J. A. Greene, A. Moiduddin. 1999. The economic impact of Staphylococcus aureus infection in New York City hospitals. Emerg Infect Dis. 5:9-17]. Additionally, antibiotic resistance is increasing among S. aureus recovered from infections [Chambers, H. F. 2001. The changing epidemiology of Staphylococcus aureus? Emerg. Infect. Dis. 7:178-181; Goetz, A., K. Posey, J. Flemming, S. Jacobs, L. Boody, M. M. Wagener, and R. R. Muder. 1999. Methicillin-resistant Staphylococcus aureus in the community: a hospital-based study. Infect. Control Hosp. Epidemiol. 20:689-691; Lowy F. D. 2003. Antimicrobial resistance: the example of Staphylococcus aureus. J. Clin. Invest. 111: 1265-1273]. Immune-directed strategies are attractive as a new frontier for developing anti-staphylococcal therapies in an era of increasing antibiotic resistance.

Complement is a critical host-defense for controlling many bacterial pathogens [reviewed in Frank, M. M. and J. P. Atkinson. 2001. Complement system, p. 281-298. In K. F. Austen, M. M. Frank, J. P. Atkinson, and H. Cantor (ed.), Samter's Immunologic Diseases. Lippincott Williams and Wilkins, New York, N.Y.] and appears to play a vital role in controlling S. aureus infections [Cunnion, K. M., D. K. Benjamin, Jr., C. G. Hester, and M. M. Frank. 2004. Role of complement receptors 1 and 2 (CD35 and CD21), C3, C4, and C5 in survival by mice of Staphylococcus aureus bacteremia. J. Lab. Clin. Med. 143: 358-365; Leijh, P. c., M. T. van den Barselaar, M. R. Daha, and R. van Furth. 1981, Participation of immunoglobulins and complement components in the intracellular killing of Staphylococcus aureus and Escherichia coli by human granulocytes. Infect. Immun. 33: 714-724; Peterson, P. K., B. J. Wilkinson, Y. Kim, D. Schmeling, and P. G. Quie. 1978, Influence of encapsulation on staphylococcal opsonization and phagocytosis by human polymorphonuclear leukocytes. Infect. Immun. 19:943-949; Verbrugh, H. A., P. K. Peterson, B-¥. T. Nguyen, S. P. Sisson, and ¥. Kim. 1982. Opsonization of encapsulated Staphylococcus aureus: the role of specific antibody and complement. J. Immunol. 129: 1681-1687; Verhoef, J., P. K. Peterson, ¥. Kim, L. D. Sabatu, and P. G. Quie. 1977. Opsonic requirements for staphylococcal phagocytosis: heterogeneity among strains, Immunol. 33:191-197].

Physiologic complement activation deposits the complement opsonins C3b and iC3b on the staphylococcal surface [Cunnion, K. M., J. C. Lee, and M. M. Frank. 2001. Capsule production and growth phase influence binding of complement to Staphylococcus aureus. Infect. Immun. 69: 6796-6803; Cunnion, K. M., P. S. Hair, and E. S. Buescher. 2004. Cleavage of Complement C3b to iC3b on the surface of Staphylococcus aureus is mediated by serum complement factor 1. Infect. Immun. 72:2858-2863; Gordon, D. L., J. Rice, J. J. Finlay-Jones, P. J. McDonald, and M. K. Hostetter, 1988. Analysis of C3 deposition and degradation on bacterial surfaces after opsonization. J. Infect. Dis. 157:697-704].

CD35, the primary receptor for C3b [reviewed in Ahern, J. M., and A. M. Rosengard. 1998. Complement receptors, p. 167-202. In J. E. Volanakis and M. M. Frank (ed.), The human complement system in health and disease. Marcel Dekker, New York, N.Y.], contributes strongly to mouse survival of S. aureus blood-stream infection, suggesting that C3b is vital for efficient opsonophagocytosis. The strength of CD35 binding to opsonized staphylococci, as assayed by immune adherence, correlates strongly with phagocytosis efficiency, supporting the importance of C3b mediated opsonophagocytosis [Cunnion, K. M., H.-M. Zhang, and M. M. Frank. 2003. Availability of complement bound to Staphylococcus aureus to interact with membrane complement receptors influences efficiency of phagocytosis. Infect. Immun. 71:656-662].

It has previously been shown that C3b is cleaved to iC3b on the S. aureus surface by the complement regulatory protein, factor I. Factor I is a serum enzyme that cleaves opsonic C3-fragments in the presence of a cofactor, such as serum complement factor H, to limit host tissue damage from uncontrolled complement activation [Brown, E. J., K. A. Joiner, T. A. Gaither, C. H. Hammer, and M. M. Frank, 1983. The interaction of C3b bound to pneumococci with factor H (β1H globulin), factor I (C3b/C4b inactivator), and properdin factor B of the human complement system. J. Immunol. 131:409-145; Fries, L. F., G. M. Prince, T. A. Gaither, and M. M. Frank. 1985. Factor I cofactor activity of CRI overcomes the protective effect of IgO on covalently bound C3b residues. J. Immunol. 135: 2673-2679; Gaither, T. A., C. H. Hammer, and M. M. Frank. 1979. Studies of the molecular mechanisms of C3b inactivation and a simplified assay of J31H and the C3b inactivator (C3bINA). J. Immunol. 123: 1195-1204].

C3b is necessary for formation of both the alternative complement pathway C3-convertase and the C5-convertase required for activation of the terminal complement cascade [Reviewed in Lambris, J. D., A. Sahu, and R. A. Wetsel. 1998. The chemistry and biology of C3, C4, and C5, p. 83-118. In J. E. Volanakis and M. M. Frank (ed.), The human complement system in health and disease. Marcel Dekker, New York, N.Y.]. Cleavage of C3b down-regulates further C3b generation by the alternative complement pathway and down-regulates C5a generation, a potent promoter of neutrophil chemotaxis and secondary granule release [Reviewed in Morgan, B. P. 1995. Physiology and pathophysiology of complement: progress and trends. Crit. Rev. Clin. Lab. Sci. 32:265-298].

It is an object of the present invention to provide a method and composition for inhibiting the factor I-mediated cleavage of C3b thereby enhancing efficient opsonophagocytosis of S. aureus and improving staphylococcal survival against the immune system.

### SUMMARY OF THE INVENTION

The above and other objects are realized by the present invention, one embodiment of which relates to a method for enhancing the immune response of a patient to Staphylococcus aureus infection comprising administering to the patient an effective amount of agent that inhibits the cofactor that mediates (1) the removal of C3b by factor I from Staphylococcus aureus cells to which it has bonded and/or 2) the cleavage of C3b by factor I to forms inactive as opsonins for Staphylococcus aureus cells.

Other objects, features and advantages of the present invention will become apparent to those skilled in the art from the following detailed description. It should be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1B: Phagocytosis efficiency for S. aureus opsonized with 2% NHS with anti-factor I antibody or control immunoglobulins, anti-BSA and IVIg. Phagocytosis efficiency was calculated as the fold-increase for S. aureus opsonized with NHS compared with S. aureus opsonized with heat-inactivated NHS (8NHS) and determined for both percent of PMN phagocytosing bacteria (1A) and number of bacteria phagocytosed by 100 PMN (1B). Anti-factor I increased percent phagocytosis compared with anti-BSA (P=0.048) and compared with IVIg (P=0.008). Anti-factor I increased number of bacteria phagocytosed compared with anti-BSA (P=0.017) and IVIg (P=0.016). Data are means of results of independent experiments. Error bars denote standard errors of the means.

FIGS. 2A-2C: C3-fragment deposition on S. aureus by 2% NHS with anti-factor I antibody or control immunoglobulins, anti-BSA and IVIg. Total C3-fragment binding to S. aureus was measured by C3 ELISA (2A) and iC3b bound to S. aureus was measured by iC3b ELISA (2B). No difference was found for C3-fragments bound in the presence of anti factor I compared with anti-BSA (P=0.41) or IVIg (P=0.11). Anti-factor I decreased iC3b generated on S. aureus compared to IVIg (P=0.017). Western blot analysis of C3 fragments bound to S. aureus (2C) shows minimal C3-fragment binding for heat inactivated serum and suggests diminished iC3b generation in the presence of anti-factor I compared to anti-BSA or IVIg. Data for ELISA experiments are means of independent experiments. Error bars denote standard errors of the means.

FIGS. 3A-3B: Phagocytosis efficiency of rrud-logarithrnic phase S. aureus opsonized by purified classical pathway components to coat bacteria with C3b and then incubated with factor H, factor I, or both. Phagocytosis efficiency was measured as percent of PMN phagocytosing bacteria (3A) and as number of bacteria phagocytosed by 100 PMN (3B). Factor I alone decreased percent phagocytosis compared with untreated C3b-coated S. aureus (P=0.039). Factors H and I together decreased percent phagocytosis compared with untreated C3b-coated S. aureus (P=0.001), but this was not different compared with factor I alone (P=0.20). Factor I alone decreased number of bacteria phagocytosed compared with untreated C3b-coated S. aureus (P=0.046). Factors H and I together decreased number of bacteria phagocytosed compared with untreated C3b-coated S. aureus (P=0.022), but this was not different compared with factor I alone (P=0.56). Data are means of independent experiments. Error bars denote standard errors of the means.

FIGS. 4A-4C: C3-fragments bound to C3b-coated S. aureus with factor H, or factor I, or both. The amount of iC3b bound as measured by iC3b ELISA (4A) was not changed with H, but increased with factor I (P=0.012) compared to untreated C3b-coated bacteria. The amount of iC3b bound was not statistically different with factors H and I together compared with untreated C3b-coated bacteria (P=0.186). The total amount of C3 fragments bound as measured by C3 ELISA (4B) was decreased with factor I (P=0.041) or with factors H and I together (P=0.025) compared with untreated C3b-coated bacteria. Factors H and I together was not statically different compared with factor I alone (P=0.32). Western blot analysis of bound C3-fragments (4C) suggests that treatment with factor I, or factors H and I together, decreases the amount of C3b bound and increases the amount of iC3b bound. Data are means of independent experiments. Error bars denote standard errors of the means.

FIGS. 5A-5B: C3-fragments released from the surface of C3b-coated S. aureus after incubation in buffer, factor H, factor I, or both. C3 ELISA measurements of C3-fragments released (5A) compared with incubation in buffer showed increased shedding during incubation with factor I (P=0.05) or incubation with factors H and I together (P=0.03), but not after incubation with factor H alone (P=0.09). Western blot analysis of released C3 fragments (5B) shows that in the presence of factor I, or factors H and I together, iC3b (or possibly C3c) are the predominant forms shed.

### DETAILED DESCRIPTION OF THE INVENTION

The present invention is predicated on the discovery that factor I-mediated cleavage of C3b decreases the amount of C3b bound to the S. aureus bacterial surface available for interaction with CD35, thus inhibiting efficient opsonophagocytosis and improving staphylococcal survival against the immune system. It has been discovered that, by inhibiting the cofactor with an antibody or medication, the immune response to S. aureus will be more effective thereby improving eradication of infection and human survival.

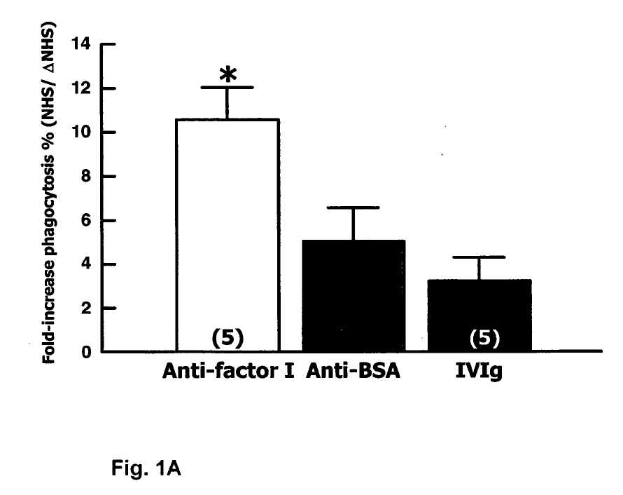

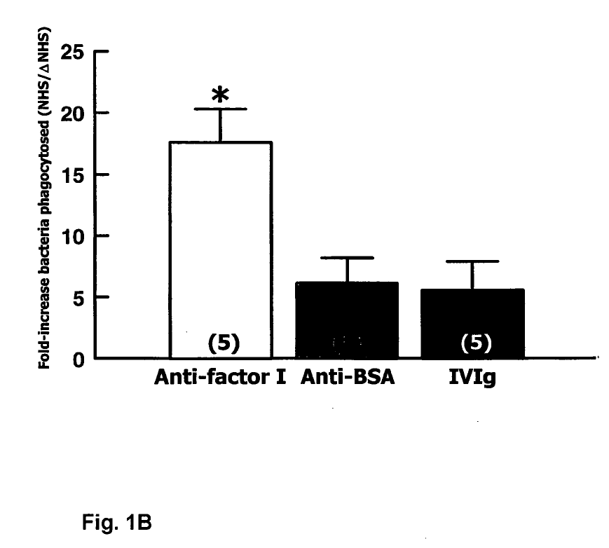

To determine whether inhibiting factor I activity would change phagocytosis efficiency, S. aureus was opsonized in 2% NHS with anti-factor I or control immunoglobulins (anti-BSA and IVIg) and the fold increase in phagocytosis efficiency compared to S. aureus opsonized in heat-inactivated (ANHS) serum with the same immunoglobulins was determined. The percent of PMN phagocytosing bacteria (FIG. 1A) when factor I was bound by anti-factor I antibody was increased 5-fold compared to anti-BSA treatment (p=0.048) and 7-fold compared to IVIg treatment (P=0.008). Mean fold-increases in the number of bacteria phagocytosed (FIG. 1B) when factor I was bound with anti-factor I were 11-fold compared to anti-BSA (P=0.017) and 12-fold compared to IVIg (P=0.016). These findings show that binding of factor I with anti-factor I increased phagocytosis efficiency of S. aureus in the presence of complement activation and demonstrate that phagocytosis efficiency may be improved by inhibiting factor I-mediated cleavage of C3b to iC3b.

### EXAMPLE

Bacterial strains and growth. Staphylococcus aureus strain Reynolds, capsule polysaccharide serotype 5, was used in all experiments. This strain has a pattern of C3-fragment deposition similar to other capsule polysaccharide serotype 5 and 8 strains—the serotypes that account for the majority of human S. aureus infections. Mid-logarithmic phase bacteria were grown in Columbia 2% NaCl broth at 37° C. with agitation for 2 hours to an optical densitometry of 0.7 to 1.3 at 600 nm. In midlogarithmic phase growth minimal capsule is expressed by strain Reynolds.

Complement buffers. Serum complement activation experiments were performed with GVBS⁺⁺ buffer (Veronal-buffered saline [VBS] with 0.1% gelatin, 0.15 mM CaCl₂, and 1.0 mM MgCl₂). Classical complement pathway activation with purified components was conducted with 60% DGVBS⁺⁺ buffer (60% VBS with 3% dextrose, 0.1% gelatin, 0.15 mM CaCl₂, and 1.0 mM MgCl₂). Complement activation was halted with EDTA-GVBS⁻⁻ buffer (VBS with 0.1% gelatin, and 0.01 M EDTA).

Complement and immunoglobulin sources. Serum for complement activation was obtained from the blood of healthy human volunteers in accordance with an Institutional Review Board-approved protocol (Eastern Virginia Medical School IRB number 02-06 EX-0216.) Blood was collected into sterile glass tubes without additives, maintained at room temperature for one hour and on ice for two hours to allow clotting. The clot was sedimented by centrifugation to recover normal human serum (NHS). The serum of four individuals was pooled and frozen at −80° C. as a stock used for all experiments. Immunoglobulin sources were commercially obtained including human immunoglobulin (IVIg) Gamimmune N (Miles Inc., Elkhart, Ind.), goat anti-human factor I (Advance Research Technologies, San Diego, Calif.), and goat anti-bovine serum albumin (Sigma Aldrich, St. Louis, Mo.). Purified human complement proteins C1, C2, C3, C4, factor H, and factor I were purchased commercially (Advance Research Technologies, San Diego, Calif.). The purity and functional activity of the purified factor H and factor I had been previously tested.

Adsorbed immunoglobulin for opsonization. Bacteria grown to mid-logarithmic phase in Columbia broth with 2% NaCl were washed twice with PBS and suspended to 1×10⁹ cells/ml in PBS with 2.5% glutaraldehyde. The bacteria were incubated for 1 hour at room temperature to fix the S. aureus, and then washed five times in PBS. The bacteria were suspended with either goat anti-human factor I, goat anti-bovine serum albumin, or IVlg at 4.2 mg of immunoglobulin with 7.5×10⁸ cells for 10 minutes. Bacteria were sedimented and the immunoglobulin was incubated with a new aliquot of staphylococci twice more.

Opsonization with serum and immunoglobulin. Mid-logarithmic phase bacteria were washed with GVBS⁺⁺ buffer and diluted to a standard concentration in the same buffer by optical densitometry at 600 nm. 2% NHS in GVBS⁺⁺ buffer was incubated with 4.2 mg of either anti-factor I antibody, anti-bovine serum albumin (BSA) antibody, or IVlg on ice for 30 minutes. Staphylococci at 1×10⁸ CFU/ml were then incubated with the serum and antibody mixtures for 30 min. at 37° C. with agitation. Negative controls were generated incubating bacteria with heat-inactivated serum and immunoglobulin. The bacteria were then washed twice with EDTA-GVBS buffer and suspended in Hanks balanced salt solution (HBSS).

Opsonization with purified complement components. Washed bacteria (1×10⁹ CFU in GVBS⁺⁺ buffer) were incubated with 0.35% IVIg for 30 min. at room temperature in order to sensitize the bacteria for activating the classical complement pathway. Antibody-coated bacteria were then incubated in 60% DGVBS⁺⁺ buffer with the purified human complement proteins C1, C4, C2, and C3 to generate the classical complement pathway C3-convertase and bind C3b to the S. aureus surface. Bacteria were incubated with C1 (2 μg/ml) at 30° C. for 15 min., and then with C4 (10 μg/ml) at 37° C. for 45 min. Bacteria were then incubated with C2 (0.5 μg/ml) and C3 (10 μg/ml) together at 30° C. for 30 min. C3b-coated staphylococci were then incubated with factor H (40 μg/ml), factor 1 (4 μg/ml), or both factors for 30 min. at 37° C. The bacteria pellet was then sedimented and the supernatants recovered to measure C3-fragments released from the bacteria. The bacteria were then washed and treated with 0.05 ml of 25 mM methylamine (Sigma-Aldrich) for 60 min. at 37° C. to recover C3-fragments bound by ester bonds to the S. aureus surface. The stripped bacteria were sedimented and the supernatants containing previously surface bound C3-fragments were recovered for testing by ELISA and Western blot. Calculation of the percentage of C3-fragments shed was performed by the following formula [C3-fragments shed/(C3-fragments shed+residual surface bound C3-fragments)].

Phagocytosis assay. Human polymorphonuclear leukocytes (PMN) were prepared from heparinized human blood from healthy human volunteers by Hypaque-ficoll step gradient centrifugation, dextran sedimentation, and hypotonic lysis. For serum opsonized-bacteria: 1×10⁶ PMN were incubated with 2×10⁷ bacteria and 1 μM N-formyl-Met-Leu-Phe (Sigma-Aldrich), in order to stimulate neutrophil phagocytosis, in a total of 1.0 ml HBSS and tumbled for 45 min. at 37° C. For purified complement component opsonized-bacteria: 1×10⁶ PMN were incubated with 1×10⁸ bacteria and 10 μM N-formyl-Met-Leu-Phe in a total of 0.5 ml HBSS and tumbled for 45 min. at 37° C. A 100 μl aliquot of the mixture was removed, stained with acridine orange (0.01% final) for 2 min., quenched with crystal violet (0.03% final) for 5 min., and then fixed to a microscope slide by cytospin. Bacteria and PMN were visualized by fluorescence microscopy using broad wavelength excitation and emission filters that allow visualization at 100× magnification. Microscope slides were blinded to the reader, and 100 neutrophils per slide were counted to determine the percent of PMN phagocytosing bacteria and total number of S. aureus ingested.

Analysis of surface-bound and released C3-fragments. ELISA was used to quantitate total C3-fragment amount and iC3b amount as follows. Flat bottom Immulon 2 plates were coated with goat anti-human C3 (Advanced Research Technologies, San Diego, Calif.) at 10 μg/ml in a carbonate coating buffer overnight at 4° C. Plates were washed 3 times (PBS, 0.1% Tween 20) and blocked (3% BSA in the same buffer) overnight at 4° C. At the time of use, plates were washed 3 times with PBS/Tween buffer and then incubated with test samples for 1 hour at room temperature. Dilutions of purified C3 or purified iC3b (Advanced Research Technologies, San Diego, Calif.) were used to generate standard curves. For the C3 ELISA: plates were washed 3 times with PBS/Tween buffer, incubated with 1:1,000 rabbit anti-human C3 antibody (Serotec, Raleigh, N.C.), washed 3 times, and incubated with 0.66 μg/ml goat anti-rabbit antibody horseradish peroxidase-conjugate (Accurate Chemical and Scientific Corporation, Westbury, N.Y.) for 1 hour at room temperature. For the iC3b ELISA: plates were washed 3 three times with PBS/Tween buffer, then incubated with 2.75 μg/ml mouse anti-human iC3b (Quidel, San Diego, Calif.) that recognizes an iC3b neoantigen for 1 hour at room temperature. Plates were washed 3 times with PBS/Tween buffer and then incubated with 1.1 μg/ml goat anti-mouse horseradish peroxidase-conjugate (Sigma Aldrich) for 1 hour at room temperature. Plates were washed 3 three times with PBS/Tween buffer and developed with TMB Plus (Accurate Chemical), stopped with 2.5 N H₂SO₄, and read at 450 nm.

Values for C3-fragments bound and iC3b bound to S. aureus opsonized in serum were calculated subtracting the values obtained with heat inactivated serum from the values obtained with NHS. Methylamine supernatants of surface bound C3-fragments were analyzed by Western blot analysis performed with polyclonal goat anti-human C3 antibody (Advanced Research Technologies) and horseradish peroxidase-labeled rabbit anti-goat antibody (Sigma-Aldrich). Bound antibody was detected by enhanced chemiluminescence.

Phagocytosis efficiency with factor I inhibition. To determine whether inhibiting factor I activity would change phagocytosis efficiency, S. aureus was opsonized in 2% NHS with anti-factor I or control immunoglobulins (anti-BSA and IVIg) and the fold increase in phagocytosis efficiency compared to S. aureus opsonized in heat-inactivated (ΔNHS) serum with the same immunoglobulins was determined. The percent of PMN phagocytosing bacteria (FIG. 1A) when factor I was bound by anti-factor I antibody was increased 5-fold compared to anti-BSA treatment (P=0.048) and 7-fold compared to IVIg treatment (P=0.008). Mean fold-increases in the number of bacteria phagocytosed (Fig. IB) when factor I was bound with anti-factor I were 11-fold compared to anti-BSA (P=0.017) and 12-fold compared to IVIg (P=0.016). These findings show that binding of factor I with anti-factor I increased phagocytosis efficiency of S. aureus in the presence of complement activation and demonstrate that phagocytosis efficiency may be improved by inhibiting factor I-mediated cleavage of C3b to iC3b.

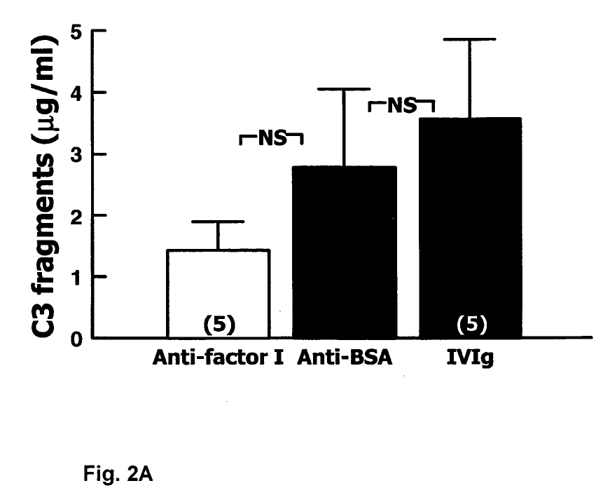

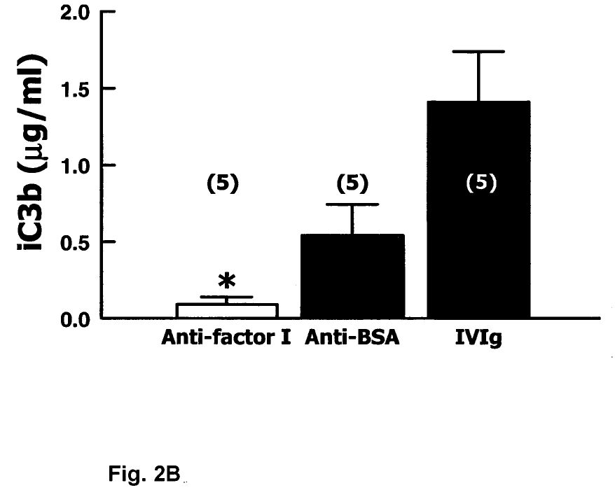

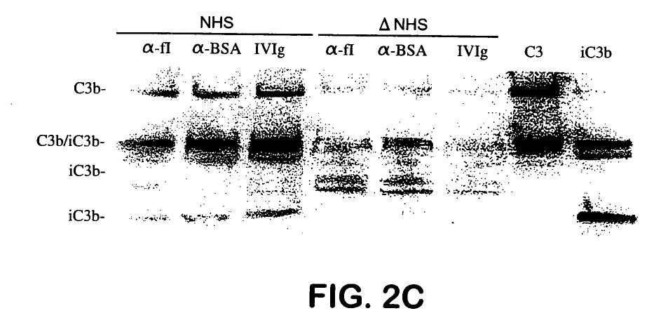

C3-fragments bound to S. aureus with factor I inhibition. To test whether anti-factor I antibody affected factor I activity on C3b, the amounts of C3-fragments bound to S. aureus were examined by ELISA and Western blot analysis. The amount of total C3 fragments bound to S. aureus in the presence of anti-factor I antibody (FIG. 2A) was not statistically different from the amount of C3-fragments bound in the presence of anti BSA antibody (P=0.41) or IVlg (P=0.11). This indicates that the increased phagocytosis efficiency in the presence of anti-factor I antibody is not due to increased C3-fragment binding to S. aureus. In contrast, the amount of iC3b bound to S. aureus in the presence of anti-factor I antibody (FIG. 2B) decreased 94% compared to the amount of iC3b bound in the presence of IVIg (P=0.017). Similarly, the amount of iC3b bound to S. aureus in the presence of anti-factor I antibody decreased 83% relative to the amount of iC3b bound in the presence of anti-BSA antibody, although this effect did not reach statistical significance (P=0.12). Anti-factor I antibody decreased the amount of iC3b bound to the S. aureus surface demonstrating that the anti-factor I antibody inhibited factor I-mediated cleavage of C3b to iC3b, leaving more C3b available as an opsonin. Western-blot with polyclonal anti-C3 antibody (FIG. 2C) showed fewer iC3b fragments were bound to bacteria in the presence of anti-factor I antibody.

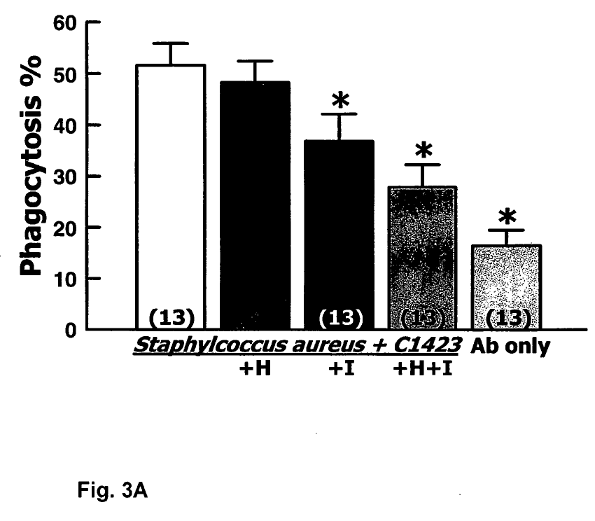

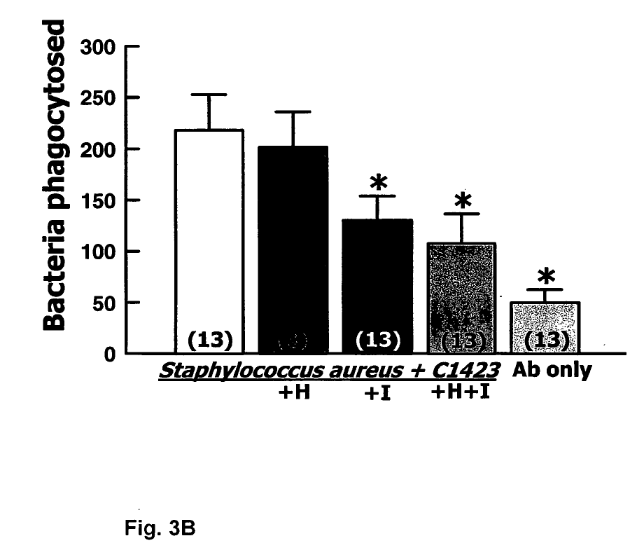

S. aureus phagocytosis with factor H and factor I. To examine how factor H, factor I, or both would affect S. aureus phagocytosis, mid-logarithmic phase bacteria were coated with C3b using purified components and then exposed to purified preparations of serum regulators of complement. The percent of neutrophils phagocytosing C3b-coated S. aureus (FIG. 3A) did not change with factor H exposure alone (P=0.58), but decreased by 29% with factor I only (P=0.039), and by 46% with factor H and factor I together (P=0.001). The percent of neutrophils phagocytosing C3b-coated S. aureus was not statistically different comparing factor I alone with factor H and factor I together (P=0.20). The number of C3b-coated S. aureus phagocytosed by 100 neutrophils (FIG. 3B) was constant with factor H alone (P=0.73), but decreased by 40% with factor I alone (P=0.046), and by 50% with factor H and factor I together (P=0.022). The number of C3b-coated S. aureus phagocytosed by 100 neutrophils was not statistically different comparing factor I alone with factors H and I together (P=0.55). These findings show that the phagocytosis efficiency of C3b-coated S. aureus is decreased by incubation with factor I, but not factor H. Notably, phagocytosis efficiency was not further enhanced when factor H and factor I were present together. These data demonstrate that phagocytosis efficiency was decreased by factor I-mediated cleavage of C3b to iC3b.

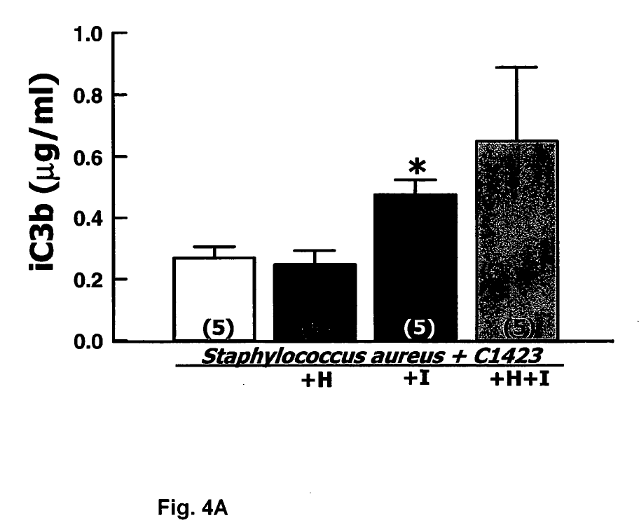

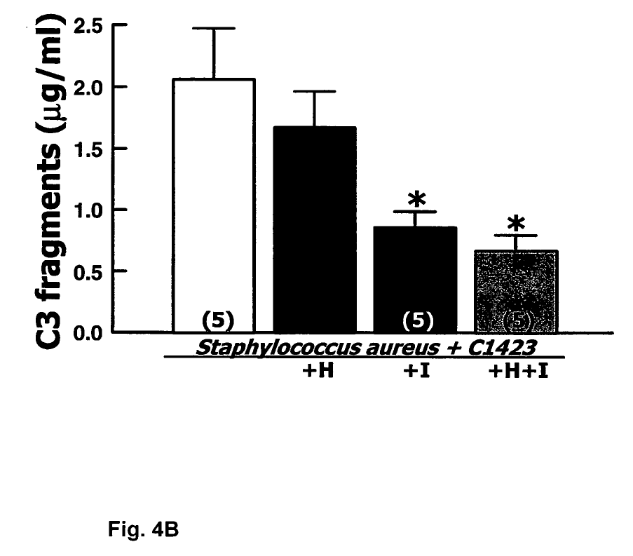

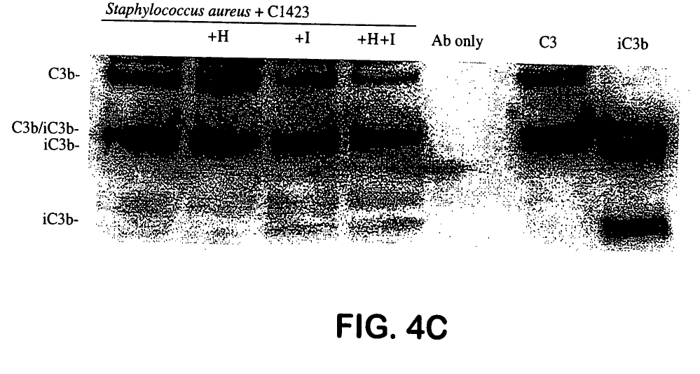

Effects of factors H and I on C3-fragments bound to S. aureus. To test whether factor H, factor I, or both, changed the C3-fragment types or amounts bound to C3b coated S. aureus opsonized by the classical pathway using purified complement components, iC3b and total C3-fragment ELISAs were performed. The amount of iC3b bound to S. aureus (FIG. 4A) was unchanged in the presence of factor H, but increased 75% with factor I (P=0.012) compared with untreated C3b-coated bacteria. Incubation with factors H and I together appeared to increase the amount of iC3b bound to S. aureus, but this did not reach statistical significance (P=0.186). These findings demonstrate that factor I was able to increase the amount of iC3b fragments on the surface of C3b-coated S. aureus by C3b cleavage.

The amount of total C3-fragments bound to C3b-coated S. aureus (FIG. 4B) was not changed in the presence of factor H (P=0.45), but was decreased by 58% with factor I (P=0.041) compared to control bacteria (no factor H or I exposure). The presence of factors H and I together decreased the total amount of C3-fragments bound to C3b-coated S. aureus by 68% (P=0.025) compared with untreated bacteria, but did not yield a different result compared with incubation with factor I alone (P=0.32). This demonstrates that factor I alone and factors H and I together can decrease the amount of C3-fragments present on the surface of C3b-coated S. aureus. Western blot analysis of C3-fragments bound to the S. aureus surface (FIG. 4C) after treatment with factor H showed no difference compared with untreated C3b-coated S. aureus. Examination of the types of C3-fragments bound to the bacteria after treatment with factor I alone or factors H and I together showed that less C3b and more iC3b were present compared with untreated C3b-coated S. aureus. These findings are consistent with findings by ELISA testing.

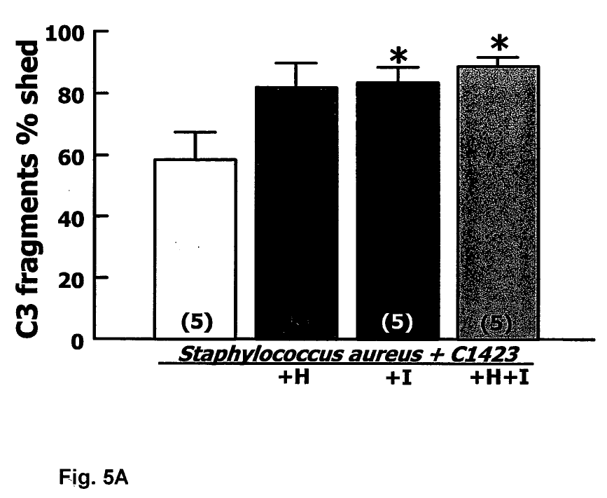

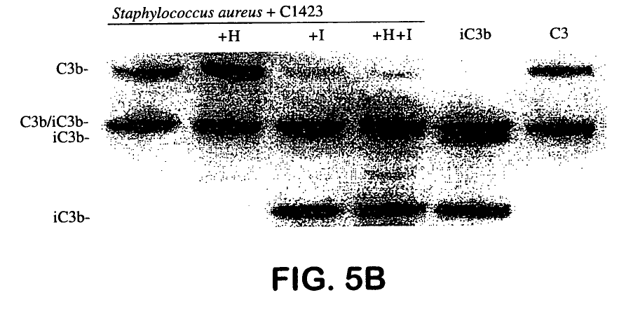

C3-fragments released from S. aureus with factor H and factor I. ELISA quantitation of the amounts of C3-fragments shed from the S. aureus surface (FIG. 5A) showed they increased significantly following incubation with factor I (P=0.05) or incubation with factors H and I together (P=0.03) compared with incubation in buffer alone. Western blot analysis with polyclonal anti-C3 antibody (FIG. 5B) showed that the C3-fragments shed after incubation with factor I or factors H and I together were likely iC3b or possibly C3c, compared with predominantly C3b being shed after incubation with buffer or factor H alone. Designed experiments were unable to detect the 22.5 kDa α′₃ fragment of C3 (i.e. C3c) either by Western blot with polyclonal anti-C3 antibody or by staining of SDS PAGE gel with Sypro Ruby a total protein stain (data not shown).

Complement plays a vital role in host defense against many bacteria, but the elements of complement-mediated control of encapsulated S. aureus remain incompletely detailed. It has previously been shown that cleavage of the important complement opsonin C3b on the S. aureus surface is mediated by factor I and could represent a staphylococcal mechanism of immune evasion. The present invention is predicated on the hypothesis that the factor I-mediated cleavage would decrease phagocytosis efficiency by reducing the number of opsonic C3b molecules bound to the S. aureus surface. Additionally, once C3b is cleaved it can no longer form the alternative complement pathway C3 convertase or the terminal complement cascade C5 convertase. S. aureus opsonized in serum in the presence of anti-factor I antibody were phagocytosed more avidly by neutrophils compared with bacteria opsonized in serum with control antibodies. This demonstrates that inhibition of factor I-mediated cleavage of C3b bound to S. aureus increased phagocytosis efficiency. The amount of iC3b bound to the bacteria is much less in the presence of anti-factor I antibody, demonstrating that the anti factor I antibody inhibited factor I-mediated cleavage of C3b to iC3b. These findings support the hypothesis that factor I mediated cleavage of C3b decreases phagocytosis efficiency.

S. aureus, opsonized using purified components of the classical complement pathway to generate C3b-coated bacteria, were then incubated in buffer, factor H, factor I, or both. The presence of factor I significantly decreased phagocytosis efficiency. Of particular note, there was no difference in phagocytosis efficiency in the presence of factor I or factors H and I together, demonstrating that this known cofactor for factor I was not necessary for factor I to decrease phagocytosis efficiency of opsonized S. aureus. The amount of iC3b on the surface of S. aureus was increased in the presence of factor I alone, suggesting that factor I cleaved C3b to iC3b on the S. aureus surface. The total amount of C3-fragments bound to the S. aureus surface was decreased in the presence of factor I suggesting that factor I cleaved some of the C3-fragments from the bacterial surface. Quantitative measurements of the amounts of C3-fragments shed from the S. aureus surface showed that increased C3-fragment shedding did occur in the presence of factor I compared with incubation in buffer alone. This is another mechanism whereby factor I was able to degrade the opsonization of S. aureus. These findings support the hypothesis that factor I mediates C3b cleavage on the S. aureus surface with resultant decreases in phagocytosis efficiency.

In summary, the above data indicate that factor I actively cleaves C3b bound to the S. aureus surface, resulting in decreased phagocytosis of these opsonized bacteria. This leads to the inescapable conclusion that factor I-mediated cleavage of S. aureus-bound C3b is a physiologically important mechanism by which the bacteria modify a vital host defense, potentially enhancing their survival. Factor I-mediated C3b cleavage does not appear to require the presence of a known cofactor. Thus, the mechanism of factor I-mediated activity on the S. aureus surface is a potential therapeutic target to enhance host defense against this pathogen.

From the foregoing description, various modifications and changes in the composition and method will occur to those skilled in the art. All such modifications coming within the scope of the appended claims are intended to be included therein. The entire disclosures and contents of each and all references cited and discussed herein are expressly incorporated herein by reference. All percentages expressed herein are by weight unless otherwise indicated.

1. A method for enhancing the immune response of a patient to Staphylococcus aureus infection comprising administering to said patient an effective amount of agent that inhibits the cofactor that mediates (1) the removal of C3b by factor I from Staphylococcus aureus cells to which it has bonded by factor I and/or 2) the cleavage of C3b by factor I to forms inactive as opsonins for Staphylococcus aureus cells.

2. The method of claim 1 wherein said cofactor is said cofactor is generated by said Staphylococcus aureus cells.

3. The method of claim 2 wherein said cofactor is contained in said Staphylococcus aureus cell walls.

4. The method of claim 1 wherein said agent is an anti-factor I antibody.

5. A pharmaceutical composition for enhancing the immune response of a patient to Staphylococcus aureus infection comprising an effective amount of agent that inhibits the cofactor that mediates (1) the removal of C3b by factor I from Staphylococcus aureus cells to which it has bonded and/or 2) the cleavage of C3b by factor I to forms inactive as opsonins for Staphylococcus aureus cells and a pharmaceutically acceptable carrier therefore.

6. The composition of claim 5 wherein said agent is an anti-factor I antibody.

7. An article of manufacture comprising packaging material and a pharmaceutical agent contained within said packaging material, wherein said pharmaceutical agent is effective for the treatment of a subject suffering from Staphylococcus aureus infection, and wherein said packaging material comprises a label which indicates that said pharmaceutical agent can be used for ameliorating the symptoms associated with Staphylococcus aureus infection, and wherein said pharmaceutical agent is one that inhibits the cofactor that mediates (1) the removal of C3b by factor I from Staphylococcus aureus cells to which it has bonded and/or 2) the cleavage of C3b by factor I to forms inactive as opsonins for Staphylococcus aureus cells.
